(19) United States
(12) Patent Application Publication
(10) Pub. No.: US 2018/0220723 A1
(43) Pub. Date: Aug. 9, 2018

(54) Orthopedic Padded Clothing System

(71) Applicant: Christopher Martin York, Goodlettsville, TN (US)

(72) Inventor: Christopher Martin York, Goodlettsville, TN (US)

(21) Appl. No.: 15/890,262

(22) Filed: Feb. 6, 2018

Related U.S. Application Data
(60) Provisional application No. 62/455,251, filed on Feb. 6, 2017.

(51) Int. Cl. A41D 13/05; A61F 5/01; A61F 5/34

(52) U.S. Cl. CPC A41D 13/0593; A61F 5/012; A63B 71/12; A61F 2005/0172; A61F 5/34

(57) ABSTRACT

An orthopedic padded clothing system including at least one garment assembly having a garment including an outer-surface, and an inner-surface having a plurality of pockets, and at least one padded insert. The plurality of pockets is/are located on the inner-surface of the garment, the plurality of pockets strategically placed on the garment such that pain and stress is reduced on at least one targeted area of a body of a user-wearer during use. The garment provides pain relief prevents discomfort, pain, stress, and injury to the body by protecting key areas.

### CROSS REFERENCE TO RELATED APPLICATION

The present application is related to and claims priority to U.S. Provisional Patent Application No. 62/455,251 filed Feb. 6, 2017, which is incorporated by reference herein in its entirety.

### BACKGROUND OF THE INVENTION

The following includes information that may be useful in understanding the present disclosure. It is not an admission that any of the information provided herein is prior art nor material to the presently described or claimed inventions, nor that any publication or document that is specifically or implicitly referenced is prior art.

### 1. FIELD OF THE INVENTION

The present invention relates generally to the field of garments and more specifically relates to therapeutic garments.

### 2. DESCRIPTION OF RELATED ART

Many people suffer from pain due to repetitive motion, weight, etc. Pain is an unpleasant sensory and emotional experience associated with actual or potential tissue damage. This may be caused by pressure points from sitting, standing, walking, running, working at a computer for an extended period of time, exercise, etc. Those working in difficult occupations are more prone to tissue damage simply from performing their duties. Many individuals, such as construction workers, find themselves performing tasks on a regular basis that are hard on the body. The knees, buttocks, and elbows are parts of the body that absorb a good deal of stress due to the force of gravity, pressure of weight and continuous motion from friction. When working, it is difficult for individuals to ensure that these areas are properly padded and protected. Many suffer from pain and injuries caused by the daily wear and tear on the body. A suitable solution is desired.

U.S. Pub. No. 2012/0005798 to Rachel N. Schatten relates to an athletic pants. The described athletic pants include an article of clothing having an outer layer, an inner layer connected to the outer layer and a cushioning insert. The inner layer is connected to the outer layer by at least two side seams. The cushioning insert is fixedly secured and is sandwiched within a predetermined area between the inner layer and the outer layer such that the cushioning insert is located between the two side seams without contacting them.

### BRIEF SUMMARY OF THE INVENTION

In view of the foregoing disadvantages inherent in the known garment art, the present disclosure provides a novel orthopedic padded clothing system. The general purpose of the present disclosure, which will be described subsequently in greater detail, is to provide articles of clothing for men and women with padded inserts.

An orthopedic padded clothing system is disclosed herein. The orthopedic padded clothing system includes at least one garment assembly having a garment including an outer-surface, and an inner-surface having a plurality of pockets, and at least one padded insert. The at least one garment assembly comprises the garment and the at least one padded insert in functional combination, each of the at least one padded insert(s) is configured to fit within confines of at least one of the plurality of pockets. The plurality of pockets are located on the inner-surface of the garment; the plurality of pockets strategically placed on the garment such that pain and stress is reduced on at least one targeted area of a body of a user-wearer during use. The garment holds the at least one padded insert against the targeted area of the body of the user-wearer, the at least one padded insert is concealed within the garment. The inner-surface is adjacent to the body of the user-wearer during an in-use condition. The orthopedic padded clothing system may provide comfort and pain relief from weight, continuous motion, and friction. The garments may have gel inserts situated in the knee, buttocks, and elbow areas. Further, the at least one garment may be used in medical institutions to help alleviate bed sores from lying in one position for too long.

For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and methods of use for the present disclosure, an orthopedic padded clothing system, constructed and operative according to the teachings of the present disclosure.

FIG. 1 is a perspective view of the orthopedic padded clothing system during an ‘in-use’ condition, according to an embodiment of the disclosure.

FIG. 2 is a perspective view of the orthopedic padded clothing system of FIG. 1, according to an embodiment of the present disclosure.

FIG. 3 is a perspective view of the orthopedic padded clothing system of FIG. 1, according to an embodiment of the present disclosure.

FIG. 4 is a perspective view of the orthopedic padded clothing system of FIG. 1, according to an embodiment of the present disclosure.

FIG. 5 is a kit of the orthopedic padded clothing system, according to an embodiment of the present disclosure.

The various embodiments of the present invention will hereinafter be described in conjunction with the appended drawings, wherein like designations denote like elements.

### DETAILED DESCRIPTION

As discussed above, embodiments of the present disclosure relate to garments and more particularly to an orthopedic padded clothing system as used to provide an efficient and effective orthopedic garment.

Generally, the present invention provides articles of clothing for men and women that include inserts filled with gel, air, foam, or other substance. It offers an orthopedic padded clothing system that provides comfort and pain relief from weight, continuous motion, and friction. It includes inserts positioned in the knee, buttocks, and elbow areas of the garments. It affords the medical community with a solution for alleviating bed sores caused from lying in one position for too long.

The present invention prevents discomfort, pain, stress, and injury to the body by protecting key areas. Orthopedic padded clothing system is designed to alleviate pain and stress on the body when working or when lying immobile in bed for long periods of time. The invention is comprised of a clothing line that includes various types of garments, such as shirts, pants, jumpsuits, and more. These garments may be commonly used clothing items in the field of construction or other labor jobs. Medical garments may also be available for use in hospitals. The clothing items all include inserts that are filled with gel, air, foam, or other material to create a padded area. The padded areas may be placed in several locations throughout the garment. These areas are considered to be where stress/force is applied to body parts, such as the knees, buttocks, and elbows. The inserts may be sewn/attached to the interior of the garment and may not be visible on the exterior. The exact specifications may vary.

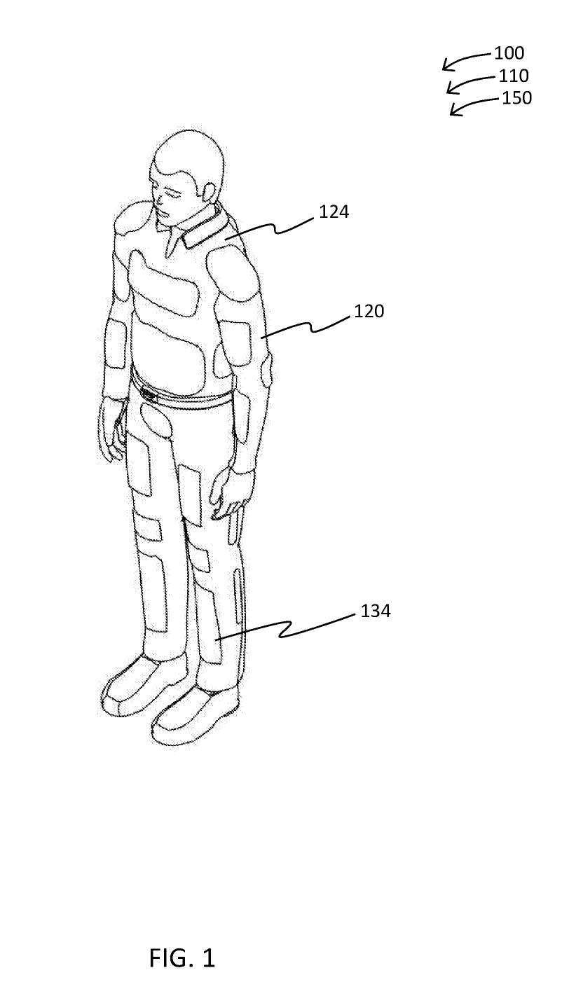

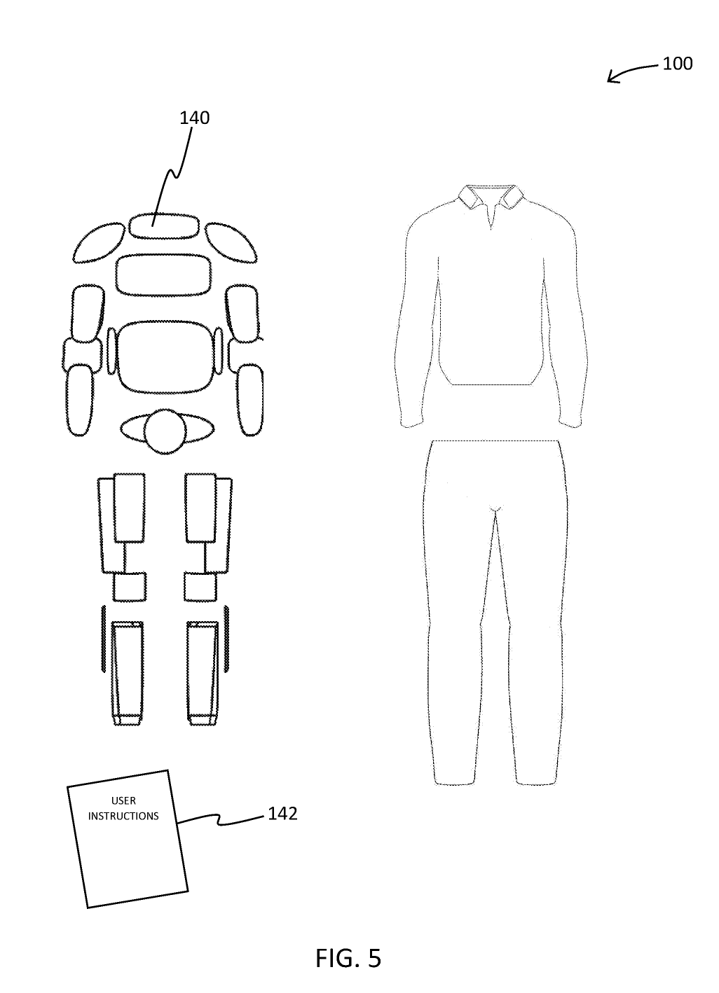

Referring now more specifically to the drawings by numerals of reference, there is shown in FIGS. 1-5, various views of an orthopedic padded clothing system 100. FIG. 1 shows an orthopedic padded clothing system 100 during an ‘in-use’ condition 150, according to an embodiment of the present disclosure. As illustrated, the orthopedic padded clothing system 100 may include at least one garment assembly 110 having a garment 120 including an outer-surface 124, and an inner-surface 130 having a plurality of pockets 134, and at least one padded insert 140. The at least one garment assembly 110 comprises the garment 120 and the at least one padded insert 140 in functional combination. Each of the at least one padded insert(s) 140 is configured to fit within confines of at least one of the plurality of pockets 134. The plurality of pockets 134 are located on the inner-surface 130 of the garment 120, the plurality of pockets 134 strategically placed on the garment 120 such that pain and stress is reduced on at least one targeted area of a body of a user-wearer during use. The garment 120 holds the at least one padded insert 140 against the targeted area of the body of the user-wearer. The at least one padded insert 140 is concealed within the garment 120. The inner-surface 130 is adjacent to the body of the user-wearer during an in-use condition.

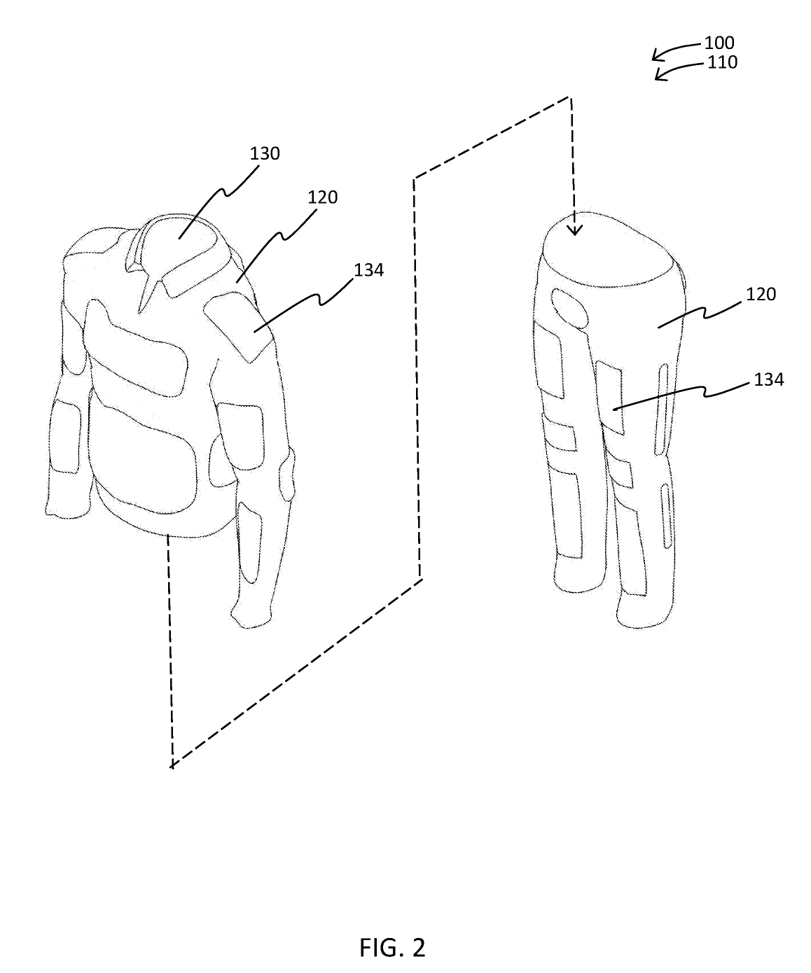

FIG. 2 shows a perspective view of the orthopedic padded clothing system 100 of FIG. 1, according to an embodiment of the present disclosure. As above, the orthopedic padded clothing system 100 may include the at least one garment assembly 110 having the garment 120 and the at least one padded insert 140. The at least one padded insert 140 may include gel, air-padding, foam, or other suitable form of padding. The at least one padded insert 140 minimizes friction on the targeted area of the body. The at least one padded insert 140 comprises a substantially similar profile to a respective one of the plurality of pockets 134 such that the at least one padded insert 140 friction-fits within one of the plurality of pockets 134 to minimize relative movement between the at least one padded insert 140 and the respective one of the plurality of pockets 134. Each of the at least one padded insert 140 is friction fit (snuggly) and compressed within a corresponding one of the plurality of pockets 134 during the in-use condition. The at least one padded insert 140 may be removeable or fastened via fastening means to the inner-surface 130. The fastening means may include sewing or other suitable fastening means.

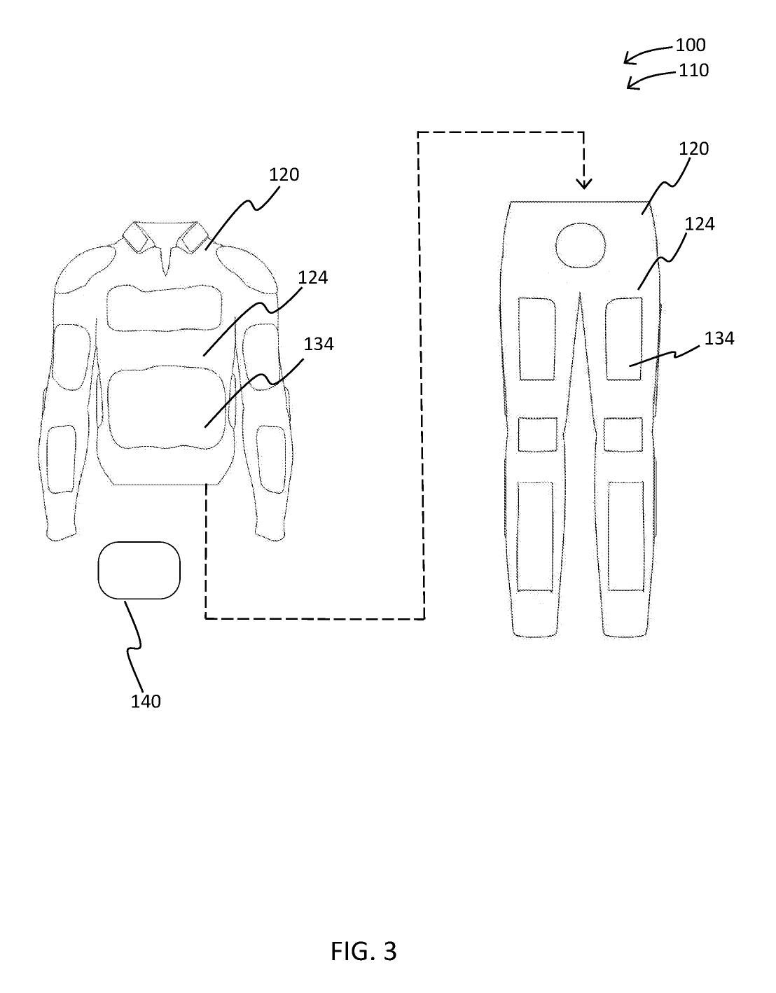

Referring now to FIG. 3 showing a perspective view of the orthopedic padded clothing system 100 of FIG. 1, according to an embodiment of the present disclosure. As above, the orthopedic padded clothing system 100 may include the at least one garment assembly 110 having the garment 120 and the at least one padded insert 140. The garment 120 is preferably unisex. The garment 120 may include a shirt, a pair of pants, a jumpsuit or a variety of other clothing items. The at least one garment assembly 110 may include a shirt and a pair of pants in combination.

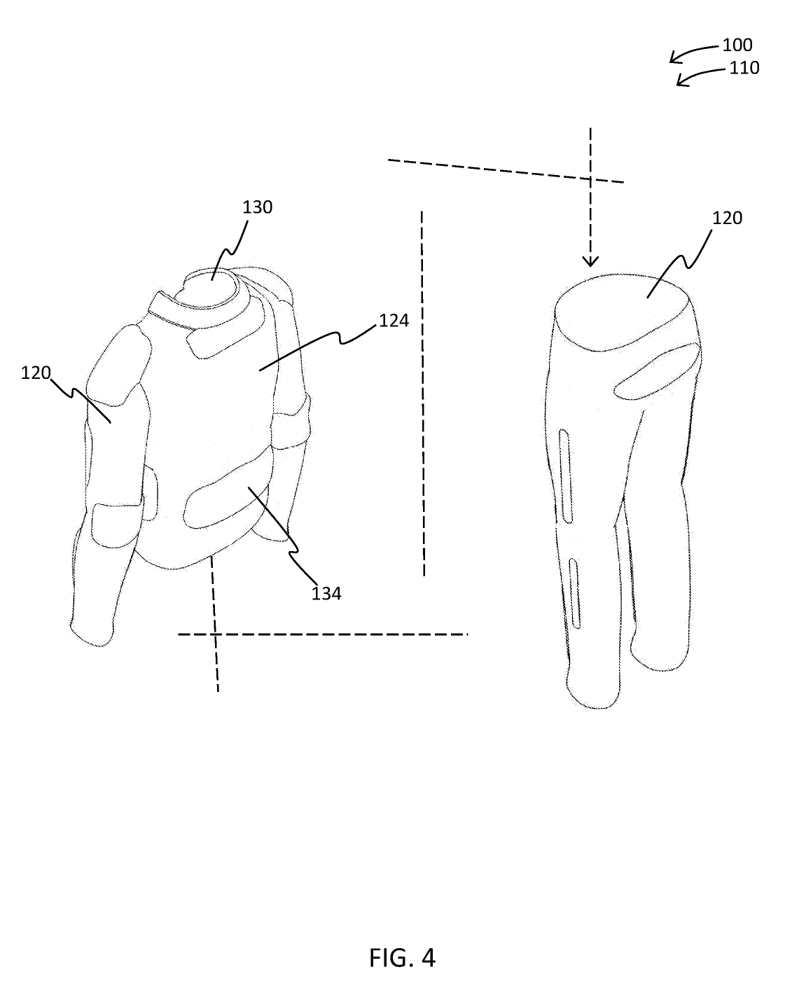

FIG. 4 shows a perspective view of the orthopedic padded clothing system 100 of FIG. 1, according to an embodiment of the present disclosure. As above, the orthopedic padded clothing system 100 may include the at least one garment assembly 110 having the garment 120 and the at least one padded insert 140 to be applied to a targeted area of the body. The targeted area of the body of the user-wearer is a joint and/or a muscle. The targeted area of the body includes but is not limited to a groin, a waistline, a buttocks, thighs, knees, shins, collar-area, shoulder, upper arm, elbows, chest, forearm, cuff-area, ribs, and abdomen. The targeted area of the body is a location of high stress as determined by the user-wearer, when the at least one padded insert 140 is applied, the high stress is reduced and pain is alleviated from the user-wearer. The at least one garment assembly 110 may be used in a medical setting to alleviate and prevent bed sores when used.

According to one embodiment, the orthopedic padded clothing system 100 may be arranged as a kit. The kit may include at least one garment having a plurality of pockets 134; at least one padded insert 140; and a set of user instructions 142. The instructions may detail functional relationships in relation to the structure of the orthopedic padded clothing system 100 (such that the orthopedic padded clothing system 100 can be used, maintained, or the like, in a preferred manner).

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. An orthopedic padded clothing system comprising:
at least one garment assembly having;
a garment including;
an outer-surface; and
an inner-surface having;
a plurality of pockets; and
at least one padded insert;
wherein said at least one garment assembly comprises said garment and said at least one padded insert in functional combination, each of said at least one padded insert(s) is configured to fit within confines of at least one of said plurality of pockets;
wherein said plurality of pockets are located on said inner-surface of said garment, said plurality of pockets strategically placed on said garment such that pain and stress is reduced on at least one targeted area of a body of a user-wearer during use;
wherein said garment holds said at least one padded insert against said targeted area of said body of said user-wearer, said at least one padded insert is concealed within the garment; and
wherein said inner-surface is adjacent to said body of said user-wearer during an in-use condition.

2. The orthopedic padded clothing system of claim 1, wherein said at least one padded insert comprises gel.

3. The orthopedic padded clothing system of claim 1, wherein said at least one padded insert uses air-padding.

4. The orthopedic padded clothing system of claim 1, wherein said at least one padded insert comprises foam.

5. The orthopedic padded clothing system of claim 1, wherein said at least one padded insert minimizes friction on said targeted area of said body.

6. The orthopedic padded clothing system of claim 1, wherein said garment is unisex.

7. The orthopedic padded clothing system of claim 6, wherein said garment is a pair of pants.

8. The orthopedic padded clothing system of claim 6, wherein said garment is a shirt.

9. The orthopedic padded clothing system of claim 1, wherein said at least one garment assembly comprises a shirt and a pair of pants in combination.

10. The orthopedic padded clothing system of claim 1, wherein said targeted area of said body of said user-wearer is a joint and a muscle.

11. The orthopedic padded clothing system of claim 9, wherein said targeted area of said body is selected from the group consisting of a groin, a waistline, a buttocks, thighs, knees, shins, collar-area, shoulder, upper arm, elbows, chest, forearm, cuff-area, ribs, and abdomen.

12. The orthopedic padded clothing system of claim 1, wherein said at least one padded insert comprises a substantially similar profile to a respective one of said plurality of pockets such that said at least one padded insert-friction-fits within one of said plurality of pockets to minimize relative movement between said at least one padded insert and said respective one of said plurality of pockets.

13. The orthopedic padded clothing system of claim 12, wherein each of said at least one padded insert is friction fit and compressed within a corresponding one of said plurality of pockets during said in-use condition.

14. The orthopedic padded clothing system of claim 13, wherein said at least one padded insert is removeable.

15. The orthopedic padded clothing system of claim 1, wherein said at least one padded insert is fastened via fastening means to said inner-surface.

16. The orthopedic padded clothing system of claim 15, wherein said fastening means includes sewing.

17. The orthopedic padded clothing system of claim 10, wherein said targeted area of said body is a location of high stress as determined by said user-wearer, when said at least one padded insert is applied, said high stress is reduced and pain is alleviated from said user-wearer.

18. The orthopedic padded clothing system of claim 1, wherein said at least one garment assembly alleviates and prevents bed sores when used.

19. An orthopedic padded clothing system comprising:
at least one garment assembly having;
a garment including;
an outer-surface; and
an inner-surface having;
a plurality of pockets; and
at least one padded insert;
wherein said at least one garment assembly comprises said garment and said at least one padded insert in functional combination, each of said at least one padded insert(s) is configured to fit within confines of at least one of said plurality of pockets;
wherein said plurality of pockets are located on said inner-surface of said garment, said plurality of pockets strategically placed on said garment such that pain and stress is reduced on at least one targeted area of a body of a user-wearer during use;
wherein said garment holds said at least one padded insert against said targeted area of said body of said user-wearer, said at least one padded insert is concealed within the garment;
wherein said at least one padded insert minimizes friction on said targeted area of said body;
wherein said targeted area of said body of said user-wearer is a joint and a muscle;
wherein said targeted area of said body is selected from the group consisting of a groin, a waistline, a buttocks, thighs, knees, shins, collar-area, shoulder, upper arm, elbows, chest, forearm, cuff-area, ribs, and abdomen;
wherein said inner-surface is adjacent to said body of said user-wearer during an in-use condition;
wherein said garment is unisex;
wherein said at least one padded insert comprises a substantially similar profile to a respective one of said plurality of pockets such that said at least one padded insert-friction-fits within one of said plurality of pockets to minimize relative movement between said at least one padded insert and said respective one of said plurality of pockets;
wherein each of said at least one padded insert is friction fit and compressed within a corresponding one of said plurality of pockets during said in-use condition;
wherein said at least one padded insert is fastened via fastening means to said inner-surface; and
wherein said targeted area of said body is a location of high stress as determined by said user-wearer, when said at least one padded insert is applied, said high stress is reduced and pain is alleviated from said user-wearer.

20. An orthopedic padded clothing system, the orthopedic padded clothing system comprising:
at least one garment having a plurality of pockets;
at least one padded insert; and
a set of user instructions.
